(19) United States
(12) Patent Application Publication
(10) Pub. No.: US 2022/0206208 A1
(43) Pub. Date: Jun. 30, 2022

(54) DEVICES FOR PRODUCING LUMINOUS DISTRIBUTIONS WITH OPTICAL WAVEGUIDES

(71) Applicant: Carl Zeiss Jena GmbH, Jena (DE)

(72) Inventors: Matthias HILLENBRAND, Jena (DE); Daniel BUBLITZ, Rausdorf (DE); Thomas NOBIS, Leipzig (DE); Martin HACKER, Jena (DE); Tobias BUEHREN, Ulm (DE); Roman KLEINDIENST, Weimar (DE); Alexander PESCH, Jena (DE)

(73) Assignee: Carl Zeiss Jena GmbH, Jena (DE)

(21) Appl. No.: 17/600,857

(22) PCT Filed: Mar. 31, 2020
(86) PCT No.: PCT/EP2020/059118

§ 371 Date: Oct. 1, 2021

(30) Foreign Application Priority Data
Apr. 3, 2019 (DE) 10 2019 108 677.9

(51) Int. Cl. F21V 8/00

(52) U.S. Cl. CPC G02B 6/0035; G02B 6/0023; G02B 6/0068; G02B 6/0016

(57) ABSTRACT

Devices for generating a luminous distribution to illuminate an object with an optical waveguide that comprises at least one input coupling element and a plurality of replication regions are provided. The device is configured to provide a luminous distribution. Further provided are a keratometer, a projection device, a microscope, a calibration device, an area lamp, and a window.

### CROSS-REFERENCE TO RELATED APPLICATIONS

This application is a national stage application, filed under under 35 U.S.C. § 371, of International Patent Application No. PCT/EP2020/059118, filed Mar. 31, 2020, which claims priority to DE 10 2019 108 677.9, filed Apr. 3, 2019, each of which is incorporated by reference herein in its entirety.

### FIELD OF THE INVENTION

The present applications relates to devices for producing illumination distributions with optical waveguides, in particular for illuminating objects.

### BACKGROUND OF THE INVENTION

In many fields of application, it can be useful to modify light provided by a light source, such that it is suitable for various applications, in particular for illuminating objects.

Waveguide systems for image generation are known from U.S. Pat. No. 8,320,032 B2 and US 2016/0231568 A1. Imaging properties in such systems need to be such that an image generated by means of a light source is preserved, which means in the case of a pixel light source, for example a pixelated display, that rays associated with a pixel that exit the waveguide at an output coupling element remain parallel to each other and thus produce a desired image on the retina when observed with a human eye. However, the present application does not relate to such arrangements with which light visualized according to data is projected accordingly, rather, it relates to the illumination of objects. It is necessary for many applications to illuminate an object, such as a sample, for example to examine the object. Such illumination is subject to various requirements regarding the illumination distribution of the lighting. It is an object to provide ways to meet such requirements.

This object is achieved by the device according to claim 1. The dependent claims define preferred exemplary embodiments.

Furthermore, a keratometer according to claim 23, a projection device according to claim 29, a microscope according to claim 31, a calibration device according to claim 34, an area lamp 38 and a window according to claim 39 are provided, each comprising such a device. Here, the dependent claims define again further preferred exemplary embodiments.

### SUMMARY OF THE INVENTION

According to the invention, a device for producing a illumination distribution for illuminating an object is provided. The device comprises an optical waveguide. The optical waveguide comprises: at least one input coupling element configured to couple light into the optical waveguide as a light beam having an associated beam profile, and

a plurality of replication regions for replication of the light beam. The plurality of replication regions are configured to receive at least one associated input light beam having an input beam profile and to provide a plurality of associated output light beams having respective output beam profiles, wherein at least one first replication region of the plurality of replication regions is optically coupled with a second replication region of the plurality of replication regions, such that the second replication region is configured to receive at least one of the plurality of associated output light beams of the first replication region as the associated input light beam of the second replication region. Here, the first replication region is optically coupled with the at least one input coupling element for receiving the light beam as the associated input light beam of the first replication region. Furthermore, the device may be configured to couple light emitted from a number of the plurality of replication regions out of the optical waveguide to provide the illumination distribution.

Here, light is understood to mean electromagnetic radiation. For example, light may be light in the visible wavelength range, but also light wholly or partly in the infrared or ultraviolet spectral range. Moreover, the methods and applications may utilize combinations of different wavelength ranges of light. For example, it may be advantageous that the illumination distribution comprises a first illumination distribution and a second illumination distribution, whereby the first illumination distribution is visible to the human eye and the second illumination distribution is not visible to the human eye.

Herein, illumination distribution is understood to refer to the light field provided by the device based on the light coupled into the device.

At least a portion of the replication regions may be configured to couple light out of the optical waveguide.

The replication regions are configured to receive at least one associated input light beam having an input beam profile. For example, individual replication regions may be configured to receive exactly one associated input light beam. In other examples, a replication region may be configured to receive several input light beams. These may in turn be provided by a single replication region or by other elements as well, for example by an input coupling element and two different replication regions.

By means of the replication regions, desired illumination distributions may be provided for illuminating, e.g., objects such as samples, which may meet various requirements. Objects may also be spatial areas such as rooms.

Input and output light beams may have different shapes, and may both be spatially sharply delimited and continuously shaped. For example, they may have rectangular, square, circular, hexagonal, or elliptical cross-sectional shapes, but other shapes and combinations of such shapes are also possible.

The optical elements mentioned above may here be diffractive elements. Diffractive elements may be conventional diffractive elements, such as kinoforms or surface gratings, but also other diffractive elements are possible, such as volume holograms. The optical elements may be fabricated differently and may be combined in the device. Other examples of possible optical devices are surface gratings, prisms, optical elements with gradient progressions of the refractive index, sometimes also referred to as gradient index optical elements, for instance known as available from the company Grintech, volume holograms, (partially mirrored) mirror surfaces embedded into the optical waveguide including Fresnel surfaces, plasmonic surfaces, and meta surfaces. Depending on the specific application, optical elements for specific fields of vision in specific wavelength ranges, for example when looking along a surface normal of the optical waveguide in the visible wavelength range of the human eye, may be either transparent, partially transparent, or intransparent.

The functionality of the optical elements that are installed and/or embedded into the optical waveguide may be supplemented by additional optical elements located outside or on top of the optical waveguide, such as mirrors, prisms, lenses, microlenses, arrays of microlenses, etc.

Some of the optical elements may be formed within the optical waveguide. This is sometimes also referred to as “writing.” For example, diffractive structures may be produced within or on the optical waveguide by means of laser writing, however, other processes are possible as well.

Here, a kinoform refers to a diffractive element with a periodic height profile. The periodic height profile may be a sawtooth profile, for example.

Replication regions may here be embodied as discrete elements separate from one another. However, replication regions may also be formed continuously, for example in regions having spatially variable optical properties. Alternatively or in addition, replication regions may overlap as well, for example when embodied as holograms in the optical waveguide, for instance by means of multiple exposure of a particular location of the optical waveguide, for example in order to generate multiply exposed volume holograms.

The device may be used to provide continuous and discrete illumination distributions. For example, one or more of fixation marks, adjustment marks, and other patterns may be provided as a illumination distribution my means of the device.

This may allow to provide compact illumination systems, for example for keratometrics, that is, the measurement of a cornea shape. Fixation marks and patterns may be used advantageously in ophthalmic devices.

Patterns may be realized both in the spatial domain as well as in the frequency domain of the illumination distribution.

Herein, fixation marks may be understood to be light presented as a virtual image to the patient at a required distance. Such fixation marks may be generated in a known manner, for example by means of a mask illuminated by a light source emitting light within the visual spectral range, whereby the mask is imaged to infinity by conventional imaging optics, for example a converging lens. The virtual image is presented to the patient within his or her field of vision. In order to see the mark clearly, the patient aligns his or her eye such that the mark is imaged onto the fovea centralis, the position of sharpest vision, and thus brings his or her eye into the desired orientation such that it is possible to take a measurement.

The devices described may thus offer alternative possibilities for providing such fixation marks. The fixation marks provided according to the invention may have improved properties, for example at least one of improved imaging quality and improved light efficiency. Additionally or alternatively, the dimensions and/or complexity of the device may be improved compared to known devices for generating fixation marks.

The devices described may also be used, for example, to provide adjustment marks and/or calibration targets for optical imaging apparatus. Some of the devices described allow advantageous illumination of a microscopic sample, for example from discrete directions, for instance for illumination at variable angles.

It is often desirable for the illumination distribution to have a specific intensity distribution. For this purpose, it may be necessary to spatially vary the strength of the individual couplings, for example between the respective replication elements. For instance, a homogeneous intensity distribution of the illumination distribution may be desired. In other words, it may be desired, for example, that all of the collimated beams propagating in the direction of an object and, for example, providing the illumination distribution transport the same power. In this case, however, the number of replication regions that the light passes through before it is deflected in the direction of the object may be different, for example because some replication regions are spatially closer to the at least one input coupling element than others. If, for example, the plurality of replication regions had the same dimensions and were e.g. arranged in rectangular fashion, and if a coupling out efficiency along a direction of the respective output light beam were constant at 10%, then 10% of the light would be coupled out as emitted light in the first interaction between the radiation propagating in the optical waveguide and such a replication region, whereas 90% would propagate further as an output light beam to a further replication region and would be received there as an input light beam. In a second interaction, only 9% of the light would be coupled out as emitted light, in a third interaction 8.1% etc. It may therefore be advantageous to vary at least one of the coupling efficiency and the output coupling efficiency of the optical elements that are used to provide the illumination distribution. This may be done, for example, with regard to the desired illumination distribution and taking into account a number of couplings that have already been traversed. In the case of continuously formed replication regions, for example, propagation length may be used as a criterion.

In order to protect the waveguide system from contamination, mechanical damage, or damage by cleaning agents/cleaning processes, the device may be combined with other elements, e.g. face plates. Both variants with and without an air gap are envisioned.

The optical waveguide may be configured to receive the light having a first modulation. In this case, the device may be configured such that the illumination distribution has a second modulation, the second modulation having a greater number of extrema than the first modulation.

A modulation is understood here to mean that the intensity of light changes as a function of a variable, for example from dark to light or from light to dark. The variable may be a position, described by one or more position coordinates, for example when the light is imaged onto a screen. Here, the variable may also be normalized to the size of the screen or the dimensions of the light or the illumination distribution. Furthermore, the variable may be one or more angles, for example defined with respect to a center of the input coupling element or a center of the object, for instance in terms of at least one of an azimuth angle and a polar angle. In this case, the modulation of light may be described by a number of extrema of the intensity of the light in question. Extrema may be maxima or minima, for example the number of light-dark or dark-light transitions with respect to the variable.

The second modulation has a greater number of extrema than the first modulation if the number of extrema, for example maxima or minima of the intensity of the illumination distribution, across a variable is greater than the number of extrema of the changes in the intensity of light across another or the same variable.

Here, the first modulation may be the modulation of the light at the input coupling element.

The first and second modulations can be determined, for example, with respect to position space or in angular space. For example, the modulation with respect to spatial coordinates in a plane perpendicular to the direction of propagation of the respective light, of which the modulation is to be determined, may be selected as position space. In other examples the modulation may be determined as a function of an angle, for example when the light is not collimated, for example when the light is effectively focused towards an object.

Here, it may be useful to choose suitable ways to determine modulation depending on the type of light. For example, an angle-dependent modulation of collimated light may be difficult to determine. In such cases, a determination of the modulation in position space, for example normalized to the beam diameter or the illumination distribution, may be advantageous. A normalization can advantageously, for example, be with respect to an area e.g. of the illumination distribution or one or more characteristic lengths, for example a beam diameter or respective principal beam axes.

In the case of focused light, on the other hand, it can be advantageous to determine the modulation in angular space.

This may have the advantage that complex illumination distributions may be generated with the device without the need for a light source assembly providing the light to the input coupling element to have a high complexity of the beam profile.

A light source assembly that comprises, for example, a single LED with a collimator has, for example, a dark-light-dark modulation as a function of an angle with respect to a connecting vector between the light source assembly and an input coupling element of the at least one input coupling element. The number of extrema of such a light source assembly can be counted as a maximum or as two minima.

If the illumination distribution in an example that is not claimed also has a dark-light-dark range, the number of extrema of the second modulation is equal to the first modulation if the number of extrema is determined consistently, that is, a number of minima is compared with a number of minima or a number of maxima is compared with a number of maxima. This is common for imaging systems.

Thus, for example, a complex illumination distribution can be provided by the device from the light of a single LED. Such a complex illumination distribution can have a greater number of extrema of the modulation than the number of extrema of the modulation of the light source assembly.

For example, the illumination distribution can have a dark-light-dark-light-dark modulation as the second modulation, that is to say two maxima or three minima, respectively. In this case, the number of extrema of the second modulation is greater than the number of extrema of the first modulation. This can also be carried out accordingly for more complex light source assemblies. For example, the optical waveguide can be configured to receive light from five discrete light sources and to provide a illumination distribution that forms more than five discrete light points, for example ten light points. However, other counts of points of light greater than five and patterns other than point patterns are also possible.

In another example, a light source assembly may provide spatially essentially unmodulated light for coupling into the optical waveguide and the illumination distribution may be modulated. Essentially means here that a light source may have intrinsic—and possibly undesired—intensity modulations. For example, laser radiation may have transverse waves, sometimes referred to as transverse electromagnetic modes TEM_{xy}. However, other intensity modulations of a light source may be present as well.

In some embodiments, the device does not comprise a spatial light modulator configured to modulate, on the basis of data, light to be coupled into the optical waveguide.

Examples of such spatial light modulators are a pixelated display and a digital micromirror device (DMD). The point here is therefore not to generate an image generated by means of such a light modulation.

At least a subset of the set of the plurality of replication regions may provide a partial illumination distribution of the illumination distribution. This partial illumination distribution may have effective focusing.

This partial illumination distribution may have effective defocusing.

In the context of this application, effective focusing is understood to mean that the emitted light, which leaves the optical waveguide from an imaginary emission area, converges onto an imaginary focus area, the imaginary focus area being smaller than the emission area.

It may also be possible for the partial illumination distribution or a second partial illumination distribution to have effective defocusing. Here, the imaginary focus area may converge for a virtual beam path in a virtually continued inverse light direction, as is known, for example, from the definition of negative focal lengths for diverging lenses. The actual beam path may thus diverge for light falling onto an imaginary plane that is located away from the emission area in the direction of the beam.

At least one of the optical elements may be a volume hologram. The at least one volume hologram may be arranged straight or at an angle within the optical waveguide. The volume hologram may be exposed multiple times.

Possible gaps in the illumination distribution may be reduced by positioning a volume hologram at an angle within the optical waveguide.

Gaps in the illumination distribution can occur, for example, when light beams replicated by the replication regions have gaps in the vicinity of the optical waveguide. For example, replication regions may be arranged spatially spaced apart from one another, such that the illumination distribution, which may be provided, for example, by means of an output coupling element, may have gaps in the vicinity of the optical waveguide. Depending on the distance between the gaps and the distance to an illuminated object, gaps may also arise in the lateral illumination on the object. Such gaps may be advantageous in some applications, for example when fixation marks are provided that are to have gaps. In other applications, such gaps can be unproblematic, for example if the illumination distribution has a gap of 1 mm at the object, for example a human eye, but the object has a pupil, for example an eye pupil of the human eye, of 3 mm.

In other exemplary embodiments, for example in the case of the microscopy sample illumination, however, such gaps may be undesirable. In applications that are sensitive to such gaps, it may be advantageous to minimize gaps in the illumination distribution or to prevent them entirely.

In exemplary embodiments without volume holograms arranged at an angle, the gaps between light beams from discrete replication regions may depend on the following parameters: The shape and the diameter of the collimated beam, the size of the input coupling element, the angle of propagation of the beam after deflection by the input coupling element, for example described by a coupling angle with respect to a surface normal of the optical waveguide in the region of the input coupling element a, and the thickness of the optical waveguide. Assuming a square input coupling element with an edge length b that is completely illuminated by the collimated light beam, gaps between the light beams that form the illumination distribution can be avoided by maintaining the relationship

b/2>d*tan(α). (1)

At the same time, a has to be greater than a critical angle of the total reflection of the optical waveguide in order to ensure an almost loss-free propagation of radiation within the optical waveguide.

Thus, by appropriately designing the parameters b, d and a for such systems, gaps in the illumination distribution may be reduced or completely avoided.

By arranging volume holograms at an angle, a required thickness of the optical waveguide, which is required in order to generate a desired illumination distribution and to avoid gaps, may be reduced in some exemplary embodiments.

In other words, in the case of volume holograms arranged at an angle, it may be possible to provide illumination distributions with reduced gaps or without gaps also for optical waveguides that do not meet the condition of equation (1), for example, that have a smaller thickness d.

The device may comprise a light source assembly. The light source assembly may be configured to provide the light. The light source assembly may comprise at least one of the following elements: two light sources configured to provide light in different directions and/or on different wavelength ranges and/or to different illumination positions of the at least one input coupling element.

This enables the generation of complex light distributions, for example superpositions of illumination distributions at different wavelengths, without the need for complex light sources.

As an alternative or in addition, the light source assembly may comprise further elements, such as, for example, beam splitters, scanning mirrors, and/or a switchable element.

The plurality of replication regions may comprise a first set of replication regions, which are each optically coupled to one another. The replication regions of the first set of replication regions may each be configured to:

provide at least one first associated output beam of the plurality of output light beams to another replication region of the first set of replication regions, and to not provide at least one second associated output beam of the plurality of output light beams to another replication region of the first set of replication regions, to obtain a number of emitted beams of the first set of replication regions.

It may be possible to embody individual elements or all elements of sets of replication regions with one or more optical elements, for example as a deflection grating with low efficiency. This may be advantageous, for example, when light propagates in the optical waveguide and, due to (total) reflections, hits the one optical element in different discrete areas.

The set of emitted beams may be configured to provide the illumination distribution.

The optical coupling may have a series structure.

The optical coupling may have a tree structure.

A tree structure of the optical coupling is understood here to mean that the replication regions of the first set of replication regions each provide at least two of the plurality of associated output light beams to at least two replication regions as respective input light beams.

In one example, a replication region of the first set of replication regions may have three output light beams. In a first variant of the example, two output light beams can be provided as input light beams. Thus, a tree structure is provided which branches with 2ⁿ, where n denotes the number of replication regions. The third output light beam may be coupled out of the optical waveguide.

In a second variant of the example, all three output light beams may be provided as input light beams, whereby a 3ⁿ tree structure is formed. The light may be coupled out from further replication regions and/or separate output coupling elements or combinations thereof on branches of the tree structure.

In other examples, other branches may be provided, generally yⁿ, where y is the number of output light beams or the number of output light beams −1.

A combination of different optical couplings is also possible, for example a tree structure may first be optically coupled, and then optical elements may again be optically coupled in a series structure within a single branch of the tree structure.

The plurality of replication regions can comprise a second set of replication regions, which are each optically coupled to one another. A subset of the second set of replication regions may be configured to receive the set of emitted beams of the first set of replication regions as respective input light beams.

The optical coupling of the first set of replication regions and/or the second set of replication regions may comprise an optical coupling in series and/or the optical coupling may comprise a tree structure. In these examples, combinations of series and tree structures are possible as well.

As discussed above, at least a portion of the replication regions may be configured to couple light out of the optical waveguide. Another possibility for coupling out of light, which may be used alternatively or in addition, is described below.

The optical elements may further comprise: At least one output coupling element configured to couple light out of the optical waveguide.

This at least one output coupling element may also act as a replication region. As described above, one or more replication regions may act as an output coupling element. In other words, the functionality of replication regions and output coupling elements can coincide and/or act in a complementary fashion.

A combination of different types of coupling out is possible as well. For example, in a first region of the optical waveguide, light from one or more replication regions may be coupled out, and in a second region of the optical waveguide, light may be coupled out by one or more output coupling elements.

It is also possible for at least one output coupling element to receive light from at least two different directions, for example from at least two replication regions. The output coupling element may then provide one or more light beams as the emitted light or as a portion of the emitted light. In this case, such an at least one output coupling element may receive light with the same orientation in each case, for example portions of light beams that propagate in total reflection within the optical waveguide and are provided to the output coupling element by respective replication regions with the same orientation. The at least one output coupling element may receive several input beams with the same or similar orientation and convert them into one or more output beams in order to provide at least a portion of the illumination distribution. Here, the several output beams may have different orientations and/or wavefront shapes.

Alternatively or in addition, an output coupling element may be configured for coupling out without replication. In other words, such an output coupling element may not be optically coupled to other replication regions and/or other output coupling elements. This may be used, for example, to provide regions in the margin of the illumination distribution.

The at least one output coupling element and/or the at least one input coupling element may comprise one or more further optical elements, for example a lens, a prism, a surface grating, a polarization filter.

These optical elements may be used to further improve the quality of the illumination distribution and/or to increase the degrees of freedom for the configuration of the illumination distribution.

The illumination distribution may be configured such that a plurality of beams from different regions of the optical waveguide are emitted such that the emitted light is effectively focused and/or effectively defocused.

The plurality of beams may be collimated and/or emitted from the optical guide in discrete angular regions.

Such illumination distributions may be advantageous for some applications, for example in keratometry and microscopy.

This allows for different locations of an object, for example in the case of keratometry of the human eye, to be illuminated at different angles by collimated rays.

The at least one output coupling element may comprise at least one further optical element. This element may be configured to generate a pattern of the coupled-out light.

Such a pattern may be, for example, a line and/or a rectangle and/or a honeycomb and/or a cross shape. In other words, the optical elements that are part of the optical waveguide may be supplemented by further optical elements. For example, in one embodiment without further optical elements, light may be provided from a replication region to provide a portion of the illumination distribution. In another exemplary embodiment, the device may comprise at least one further optical element, for example a lens. In this exemplary embodiment, the light that is provided by the replication region, being emitted from the optical waveguide, may then pass through the lens and, after passing through the at least one further optical element, may contribute to the illumination distribution of the device. Here, the at least one further optical element may be formed in one piece with the optical waveguide, but it may also be configured independently thereof, for example attached on the optical waveguide or arranged with a retaining element at a distance from the optical waveguide. Functional integration of the further optical elements may also be carried out. For example, a further optical element may be part of an output coupling element, for example in that the output coupling element has a curved surface and thereby additionally acts as a converging lens.

At least one of the optical elements and/or the at least one further optical element may be a diffractive element, a switchable diffractive element, a volume hologram.

For example, the input coupling element may be embodied as a diffractive element, for example a surface grating. The first replication region may be embodied as a volume hologram. The at least one further optical element may be a switchable diffractive element. The illumination distribution provided by the device may be modified by such switchable elements.

The at least one input coupling element may be configured to carry out coupling based on a characteristic of the light. The replication regions may be configured to generate at least two different associated illumination distributions for at least two different characteristics of the light.

Characteristics of the light may be, for example, wavelength, polarization and coupling angle, position of the coupling, but also combinations thereof.

This also enables different illumination distributions to be provided by one device. By varying the light characteristics, the illumination distribution of the device may then be controlled without the device itself having to include active components. In this way, for example, a controller may modify the characteristics of the light from the light source assembly.

In yet other exemplary embodiments, different control options may also be coupled, for example optical elements which are arranged in the optical waveguide may be controllable and at the same time a characteristic of the light may be varied. In this way, illumination distributions may be changed and numerous variants of desired illumination distributions may be achieved with relatively few complex elements.

The device may be configured to provide a illumination distribution for illuminating an object remote from the device at variable angles, the object having a smaller diameter than the optical waveguide.

Here, illuminating at variable angles is understood to mean adjustable illumination from different angles and different directions.

Devices for illumination at variable angles are described, for example, in applications DE 10 2014 101 219 A1, DE 10 2016 116 31 1 A1, and DE 10 2014 112 242 A1.

In order to determine whether the object has a smaller diameter than the optical waveguide, the respective diameter of the object and optical waveguide in the direction of the illumination distribution may be taken into account. For example, the optical waveguide may also have a smaller dimension than the diameter of the object in a direction in which no illumination distribution is provided. For example, the optical waveguide may be constructed with a thickness of a few millimeters, but may have a lateral size of a few centimeters and illuminate an object with a diameter of 5 mm.

The device may be configured to provide the illumination distribution for the object, when the object is located at an angle to a surface normal of the optical waveguide.

This may have the advantage that devices have a greater degree of design freedom for a desired illumination distribution. This may, for example, offer functional and/or aesthetic advantages, for example if the device is built into a pair of glasses and is used to provide energy to an eye implant in the eye of a user. In such examples, but not limited thereto, it may be advantageous if an angle with respect to the surface normal of the optical waveguide may be selected. This may, for example, improve the aesthetics of such a pair of glasses.

According to an embodiment, an optical waveguide system with a plurality of devices is provided. The plurality of devices is realized each according to any one of the preceding exemplary embodiments, the plurality of devices having a common optical waveguide with an output coupling area. The common optical waveguide may have at least one cutout with a cutout area in the output coupling area. The plurality of devices may be arranged such that the illumination distributions of the plurality of devices originate from at least 80% of the output coupling area without the cutout area.

This may have the advantage that it may be possible to use the optical waveguide system for illumination, whereby the illumination distribution generated by the optical waveguide system can be observed by a detection system. A high degree of freedom in the illumination distribution may be achieved by the cutout, without the optical waveguide adversely affecting the detection system. This may be particularly advantageous if the optical waveguide system is to be used as an alternative illumination system for a measuring assembly and the measuring assembly is not to be qualified anew, or if the optical waveguide system is to be integrated into an existing measuring assembly, for example retrofitted. Such an optical waveguide system may also be advantageous if response light, which emanates from an object in reaction to the illumination distribution, for example, is not to be modified by interaction with an optical waveguide. In such cases, one or more cutouts may avoid undesirable effects such as refraction, reflection, loss of coherence, etc.

In keratometry, for example, a detection of the light reflected back from the cornea is required to be vignetting-free and telecentric. This may be ensured by means of a cutout between the eye and a detector.

The optical waveguide may have first and second sides. Here, the illumination distribution comprises a first illumination distribution on the first side and a second illumination distribution on the second side of the optical waveguide.

In the following, exemplary embodiments of the device described above will be described with regard to various specific possible applications. The described exemplary embodiments are to be understood as examples. The aspects of the application examples discussed below may therefore also be used in isolation from the specific possibilities of application, whether generally in devices according to the invention or in others of the described possibilities of application.

According to one application example, a keratometer for measuring the cornea of the human eye is provided.

Keratometry is a widely used method in ophthalmology and is dedicated to measuring the shape of the cornea of the human eye. The meaning and the basic functionality of this method of measurement is explained, for example, in DE 10 2011 102 355 A1. Various arrangements are known from the prior art, for example so-called Littmann keratometers, which emit collimated beams of rays from different directions onto the cornea of the eye and examine the reflected light. Here, the quality of the examination depends on the number of collimated beams. The generation of a sufficiently high number of collimated light beams is often associated with great difficulty and limits the possible quality of the method. Placido disk keratometers are based on planar or curved disks with several concentric, self-luminous rings. The reflections of the rings at the cornea may be imaged and evaluated with a camera sensor. The topography of the cornea may be determined from the deformation of the rings.

A combination of Littmann keratometers and Placido disk keratometers is known from DE 10 2011 102 355 A1. By means of a single or fewer radiation sources, an array of several collimated beams is generated, which propagate from different directions towards the pupil of the eye. By using collimated beams, the principle is more robust with regard to axial offsets of the eye, while at the same time, the large number of collimated beams makes it possible to achieve great robustness with regard to local corneal defects. In addition, the method is characterized by a high level of light efficiency. The optical element of DE 10 2011 102 355 A1 uses a combination of reflective and refractive surfaces and has a complex basic geometry, which may lead to great effort in the manufacture of the element. The diameter of the collimated beam is limited by the size of the free-form facets of the optical element used. For a sufficient tolerance of the device with respect to movement of the eye, it is necessary that the individual beams have a minimum diameter that depends on the angle of incidence. With a predetermined number of facets, this results in a large distance between the optical element and the patient's eye and, associated therewith, also a large diameter of the optical element. In the center of the element according to DE 10 2011 102 355 A1, an opening must be provided which enables vignetting-free telecentric detection of the light reflected back from the cornea. Illumination beams cannot be radiated into the region of this detection. In order to minimize the resulting measurement gap in the central corneal region, the element must be as far away from the eye as possible, which leads to larger disk diameters. The embodiments explained below make it possible to improve the various keratometers by means of the device described.

For a high measurement accuracy of an overall keratometer system, it may be advantageous to know a gradient of the beams impinging onto the eye very precisely, for example to 1/500 of the respective deflection angle with respect to a normal of the optical waveguide. The requirements for the accuracy of the corneal measurement may be reduced outside the pupil region of the eye. It may be advantageous to take into account that the geometry of the optical waveguide, the deflection behavior of the various optical elements, as well as the wavelength, position, and orientation of the light source may depend on external parameters, for example the temperature. The changing wavelength of the radiation used may in turn influence the behavior of the optical elements, for example the coupling. In addition, other factors, for example mechanical stresses induced in the optical waveguides or the force of gravity, may influence the orientation of the provided beams of rays. In order to minimize the consequences of all these effects on the measurement result and to enable the greatest possible manufacturing and assembly tolerances, a keratometer with optical waveguides may be characterized at different temperatures, humidities, and operating conditions of the light sources and this may be taken into account in an examination. Thus, by evaluating the parameters actually present during the measurement with the aid of additional sensors, for example temperature and/or humidity sensors as well as using calibration curves obtained and/or analytical or numerical models of the system behavior, a high level of measurement accuracy may be guaranteed. Such procedures may be used with the described keratometers.

The keratometer according to one embodiment comprises:

a device according to the preceding exemplary embodiments, wherein the device is configured to provide a illumination distribution comprising a plurality of collimated beams of rays with respectively defined emission regions on the optical waveguide to a human eye on a first side of the optical waveguide.

The keratometer may further comprise a detection device which is configured to receive light of the collimated beams of rays reflected by the human eye.

Due to the high spectral and angular selectivity of the device, the optical waveguide may have very good transparency despite its high efficiency when providing the illumination distribution. This may apply to all of the exemplary embodiments described here. When such devices are used in keratometry, the patient's eye may thus be observed during the measurement, for example, and it may be made easier to communicate with the patient—including eye contact. Measurement systems, for example keratometers, but also other measurement systems, where the patient may continue to observe the environment during the measurement and where the real environment and virtual illumination distributions blend, may also be possible.

The keratometer or other devices may be embodied as glasses-like measuring devices worn on the head that take measurements or consciously stimulate the patient's eye by projecting patterns during normal daily routines of the patient.

Such devices may also be used in other fields of application, for example in order to irradiate selected regions of the patient's eye for therapy, for instance with infrared radiation.

The detection device may be arranged on a second side of the optical waveguide and may be configured to receive the reflected light along a beam path through the at least one cutout.

The optical waveguide may be configured to provide a illumination distribution with a concentric ring structure. Here, the concentric ring structures may be continuous rings. However, it is also possible to provide illumination distributions with ring structures having discrete directions and/or gaps in between. Other shapes, for example ellipses, are also possible.

The concentric ring structure may have a illumination distribution similar to a Placido disk.

This may have the advantage that known examination methods may be used and at the same time the quality of the measurements may be improved and/or the complexity of the keratometer may be reduced.

The tree structure of the replication regions may be arranged along a radial direction.

This may have the advantage that the illumination distribution may have homogeneous illumination properties in a poloidal direction, for example in the case of the concentric ring structure. In this way, inhomogeneities in the illumination intensity in the poloidal direction may in particular be avoided.

A keratometer may comprise two light sources as the light source. These two light sources may be configured to provide light in different directions and/or different wavelength ranges and/or at different illumination positions of the at least one input coupling element.

Here, a first light source of the two light sources may emit light in the infrared and a second light source of the two light sources may emit light in the visible range. The output coupling element may be configured to provide the light from the second light source as fixation marks.

The infrared light may be provided, for example, by an LED and/or a laser diode and/or a superluminescent diode. The infrared light may have an intensity maximum at 825 nm or 1064 nm, for example.

Fixation marks may be patterns in the visible spectral range that originate from several positions from the optical waveguide. Fixation marks may be provided as shaped and/or collimated rays. The rays may have different directions of propagation or run parallel to one another. Combinations are also possible. The pattern can, for example, have a cross shape, but other shapes such as individual points or a single point at infinity, point patterns, empty or filled circles, empty or filled squares, etc. are also possible.

For example, a cross shape may be achieved out of five points in the illumination distribution. Here, each point may be generated by a collimated beam of rays incident on the eye. Because the five collimated beams fall onto the eye from different directions, they are focused by the eye lens at different points on the retina and generate five image points there.

The rays may emanate from virtual points in front of or behind the eye of the patient/user or converge towards these. These may be referred to as fixation marks in the finite and may be used, for example, in patients with ametropia, or to represent a near-vision target.

In some applications, complex patterns may be provided from a single collimated input beam and/or a single collimated beam within the waveguide.

The output coupling element may here be embodied, for example, as a volume hologram in order to provide the pattern in the illumination distribution, but a combination of other optical elements for generating a pattern is also possible.

The optical waveguide may comprise a response light coupling device. This device may be configured to couple the reflected light into the optical waveguide and transfer it from the optical waveguide to the evaluation device.

In this case, the response light coupling device may be designed based on a modification of a beam profile of the illumination distribution due to the reflection at the eye.

Such a response light coupling device may have the advantage that the optical structure of a detection beam path may be simplified. At the same time, more compact keratometers may be made available as a result. In other words, the flexibility of the device for generating a illumination distribution may, conversely, also be used to couple in light and transfer it to one or more detectors. Here, the path of the light may be reversed.

Such devices may have the advantage that the solid angle conventionally blocked by the detection device may be utilized differently. For example, this makes it possible to have a central view of the patient's eye. In other words, a device that conventionally appears to be a bulky optical structure may act like a window glass according to the invention. This may make the examination more pleasant. Systems worn on the head that allow a view of the surroundings are also possible.

The response light coupling device may be part of the optical waveguide. But it may also be embodied separately from the optical waveguide, for example connected to the waveguide, for example glued to the optical waveguide.

The response light coupling device may be configured to take into account a typical curvature of the wavefront. This curvature may be calculated in advance, for example, and a corresponding compensation function may be integrated into the associated input coupling elements. For this purpose, an observation beam path that takes into account the light path within the optical waveguide may be constructed telecentrically.

According to the invention, devices for projecting fixation marks and patterns in ophthalmic apparatus may be provided.

According to one exemplary embodiment, a projection device for ophthalmic apparatus is provided. The projection device comprises a device according to one of the previous exemplary embodiments, wherein the projection device may comprise a light source which may be modulated in multiple ways and which may be configured to provide the light to the device. The device may be configured to provide the illumination distribution in such a way that at least two modulated illumination distributions are provided for at least one modulation of the light source that can be modulated in multiple ways, the at least two modulated illumination distributions being modulated with respect to at least one of direction and location.

The at least two modulated illumination distributions may be marks and/or Landolt rings and/or letters and/or striped patterns, for example.

For example, the light source that can be modulated in multiple ways may have three modulations A, B, and C. Here, a illumination distribution, which is a superposition of four modulated illumination distributions, may be provided by the device for modulation A. For modulation B, the device may provide a illumination distribution that is not modulated. For modulation C, the device may provide a illumination distribution that is a superposition of two more modulated light distributions.

The direction and/or location of the at least two modulated illumination distributions may be modulated in such a way that they partially correspond and partially differ. For example, a first modulated illumination distribution may have a central cross and a peripheral ring. A second modulated illumination distribution may have the same central cross but several peripheral rings, which may be partially interrupted.

According to a further application example, a sample illumination at variable angles may be provided in a microscope.

The devices described above may be embodied as sample illumination devices, for example as an addition to an existing microscope.

According to one exemplary embodiment, a microscope with a beam path and a sample illumination device is provided. Here, the sample illumination device comprises a device according to the preceding exemplary embodiments. The optical waveguide may be configured such that, when it is arranged in the beam path, it generates a pattern on a sample in the microscope by means of the illumination distribution.

The pattern may be a switchable pattern.

The pattern may be a pattern with variable angles. The pattern may be a pattern switchable at variable angles. In other words, for each replication region or for some replication regions, the intensity ratios of the respective output light beams of the plurality of output light beams may be varied. As previously described, this may be achieved by the replication regions by themselves, or by one or more output coupling elements, or through the interaction of at least one output coupling element with the replication regions.

According to a further application example, calibration marks for optical devices are provided.

According to one embodiment, a calibration device for an optical apparatus, comprising at least one device according to preceding exemplary embodiments, is provided. The illumination distribution may be configured to provide a test light field for the optical apparatus.

The test light field may be tuned to zoom settings and/or focus plane settings and/or an installation position of the calibration device of the optical apparatus.

The installation position may be a position within optical elements of the optical apparatus and in a beam path of the optical apparatus.

According to an exemplary embodiment, a plane glass, a filter glass or a protective glass is provided for a lens. This lens may comprise a calibration device according to preceding exemplary embodiments.

According to an exemplary embodiment of a further application example, an area lamp is provided. This lamp comprises a device according to preceding exemplary embodiments. Here, the optical waveguide may have a dimension of less than 50 μm in one direction.

According to an exemplary embodiment, a window is provided. The windows comprises: a window glass comprising a device according to preceding exemplary embodiments, and a window frame comprising a light source. The light source may be configured to provide infrared light. The light source may further be configured to provide the light to the at least one input coupling element. The illumination distribution may be configured to provide the infrared light as a heat source on at least one side of the window glass.

In some exemplary embodiments, a window is attached to an outside wall of a room. In these cases, the infrared light may be provided on an inside of the window, for example in order to supply the room with heat, wherein the heat may be provided as radiant heat from the infrared light.

In another embodiment, the window is attached to an outside wall between a room and a sunroom. In this case, the illumination distribution may provide the infrared light on both sides of the window to heat both the sunroom and the room.

The infrared light may have an intensity maximum in a spectral range from 1 to 10 μm.

### BRIEF DESCRIPTION OF THE DRAWINGS

The invention is explained in detail below with reference to the drawings on the basis of exemplary embodiments:

FIG. 1A depicts a device for generating a illumination distribution in accordance with various exemplary embodiments.

FIG. 1B depicts a device according to FIG. 1A in accordance with another exemplary embodiment.

FIG. 2 depicts a front view of a device according to FIG. 1A and/or FIG. 1B.

FIG. 3 depicts an example of a device with a tree structure.

FIG. 4 depicts another exemplary embodiment of FIG. 1A and FIG. 1B.

FIG. 5 depicts a further alternative of the device of FIG. 4.

FIG. 6 depicts devices in accordance with various exemplary embodiments, which are multi-channeled and/or switchable.

FIG. 7A depicts a side view of a keratometer according to the invention.

FIG. 7B depicts a front view of the keratometer of FIG. 7A.

FIG. 8 depicts another exemplary embodiment of a keratometer.

FIG. 9 to FIG. 12 depict various implementations of keratometry devices.

FIG. 13A and FIG. 13B depict an alternative implementation of a keratometer with fixation marks.

FIG. 14 and FIG. 15 depict a microscope in accordance with various exemplary embodiments.

FIG. 16 depicts a calibration device 150 for an optical apparatus 910 in accordance with various exemplary embodiments.

FIG. 17 depicts an area lamp 170 comprising a device 100 in accordance with various exemplary embodiments.

FIG. 18 depicts a window in accordance with an exemplary embodiment.

FIG. 19 depicts an illumination device for an active eye implant in accordance with an exemplary embodiment.

### DETAILED DESCRIPTION OF THE INVENTION

In the following, various exemplary embodiments will be described in detail. These exemplary embodiments are merely for illustrative purposes and are not to be construed as limiting. For example, a description of an exemplary embodiment with a large number of elements or components should not be interpreted to the effect that all of these elements or components are necessary for implementation. Rather, other exemplary embodiments may include alternative elements or components, fewer elements or components, or additional elements or components. Elements or components of different exemplary embodiments may be combined unless indicated otherwise. Modifications and variations described for one of the exemplary embodiments may also be applicable to other exemplary embodiments.

The figures aim to illustrate the underlying principles. For example, surface shapes and refractions may be indicated schematically. Refractions may, for instance, be depicted in exaggerated fashion or neglected.

To avoid repetition, the same or corresponding elements are designated with the same reference numeral in different figures and are not explained more than once.

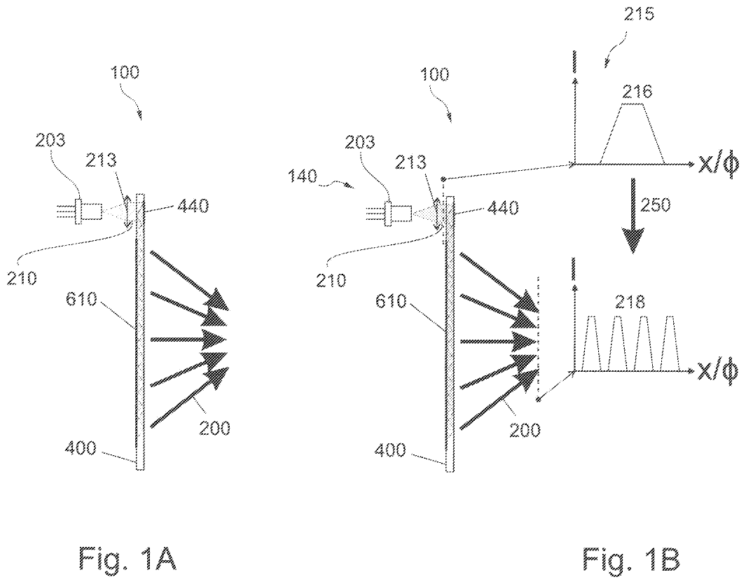

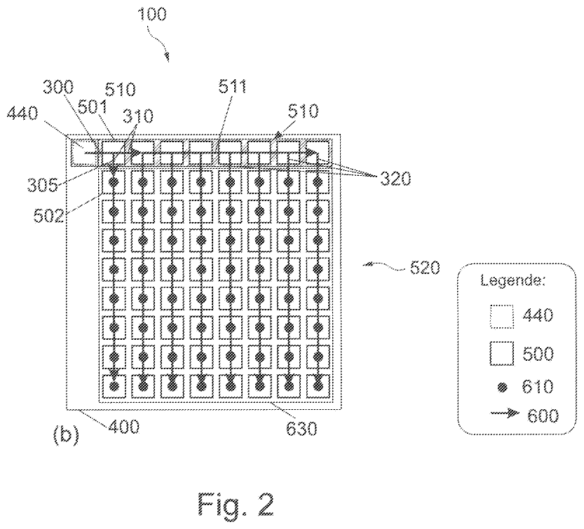

First, two exemplary embodiments of the device are explained with reference to FIG. 1A, FIG. 1B, and FIG. 2.

FIG. 1A depicts a device for generating a illumination distribution in accordance with an exemplary embodiment.

The device 100 comprises an optical waveguide 400 having an input coupling element 440. The device 100 is configured to receive light 210 from a light source 203 and emit emitted light 610 in form of a illumination distribution 200. The illumination distribution may be used to illuminate an object. In the depicted example of FIG. 1A and FIG. 1B the illumination distribution has effective focusing, which is indicated by the arrows of the illumination distribution 200 converging towards one another. In other examples, the illumination distribution may also have effective defocusing. In such cases, the arrows indicating the illumination distribution 200 would diverge and would have a virtual starting point on the left-hand side of the optical waveguide 400 in FIG. 1.

In other examples, the illumination distribution 200 may be configured such that a plurality of beams from different regions of the optical waveguide 400 are emitted such that the emitted light is effectively focused and/or effectively defocused.

For example, light from the upper half of the optical waveguide 400 could have effective focusing and light from the lower half of the optical waveguide 400 could have effective defocusing.

FIG. 1B depicts a device according to FIG. 1A in accordance with another exemplary embodiment.

In the exemplary embodiments of FIG. 1A and FIG. 1B, the light 210 is collimated by a collimator 213 before it hits the input coupling element 440. A collimated light ray may be advantageous for some input coupling elements and, for example, increase coupling efficiency. Collimation may be achieved by a separate collimator 213, as shown in the example of FIG. 1B. In other exemplary embodiments that are not shown, collimation may also be achieved by the input coupling element itself.

The light 210 has a beam profile 215 with a first modulation 216. The device 100 converts the light 210 into the illumination distribution 200. The illumination distribution 200 has a second modulation 218. Here, the number of extrema of the second modulation 218 is greater than the number of extrema of the first modulation 216. As indicated, at least one of the first and second modulations may be determined in position space (vector “x”) or in angular space (“φ”). In other words, the modulation may be observed in that the intensity is variable as a function of one or more spatial coordinates and/or as a function of one or more angular coordinates, the number of extrema for the second modulation 218 being greater than for the first modulation 216. Here, the coordinates may be normalized expediently, for example in relation to a hemisphere of a unit sphere when light is incident on an object from one side or in relation to a beam diameter in position space.

In the example of FIG. 1B, the beam profile 215 has a number of one maximum and two minima in relation to the beam diameter. On the other hand, the second modulation 218 of the illumination distribution 200 has 4 maxima and 5 minima, respectively. Thus, the second modulation 218 has a greater number of extrema than the first modulation 216.

The device can thus convert a relatively simple input light distribution into a complex output light distribution.

FIG. 2 depicts a front view of a device according to FIG. 1A and/or FIG. 1B.

In the exemplary embodiment of FIG. 2, the optical elements in the optical waveguide 400 are embodied as discrete units. In other exemplary embodiments, these may also be embodied continuously, as described above and below. an input coupling element 440 is optically coupled to a plurality of replication regions 500, the optical coupling 600 being indicated as an arrow. Each element of the plurality of replication regions 500 is configured to receive an associated input light beam having an input beam profile and to provide a plurality of associated output light beams having respective output beam profiles.

It is also possible for the light to interact several times with a replication region, for example after a total reflection within the optical waveguide.

In particular, a first replication region 501 of the plurality of replication regions 500 is optically coupled 600 to the at least one input coupling element 440, such that the first replication region 501 is configured to receive the light beam as the associated input light beam 300 of the first replication region. Furthermore, the first replication region 501 is optically coupled 600 with a second replication region 502 of the plurality of replication regions 500, such that the second replication region 502 is configured to receive one of the plurality of associated output light beams 310 of the first replication region as the associated input light beam of the second replication region 305.

The device 100 is configured to couple light 610 emitted from a number of the plurality of replication regions 500 out of the optical waveguide 400 to provide the illumination distribution 200, as depicted in FIG. 1A and FIG. 1B, for example. In this case, the light is emitted from an output coupling region 630 of the optical waveguide 400.

In the exemplary embodiment shown in FIG. 2, the plurality of replication region 500 comprises a first set of replication regions 510 (highlighted via shading), which are each optically coupled to one another and each forward the light to individual elements of the set—with the exception of the last element of the first set—and distribute it in a different direction to other replication regions that do not belong to the first set of replication regions. In the exemplary embodiment shown, all replication regions belong either to the first set of replication regions or to the number of the plurality of replication regions. In other exemplary embodiments, however, this may be varied as desired.

By means of such a device 100, the light provided at the input coupling element 440 may advantageously be converted into an illumination distribution.

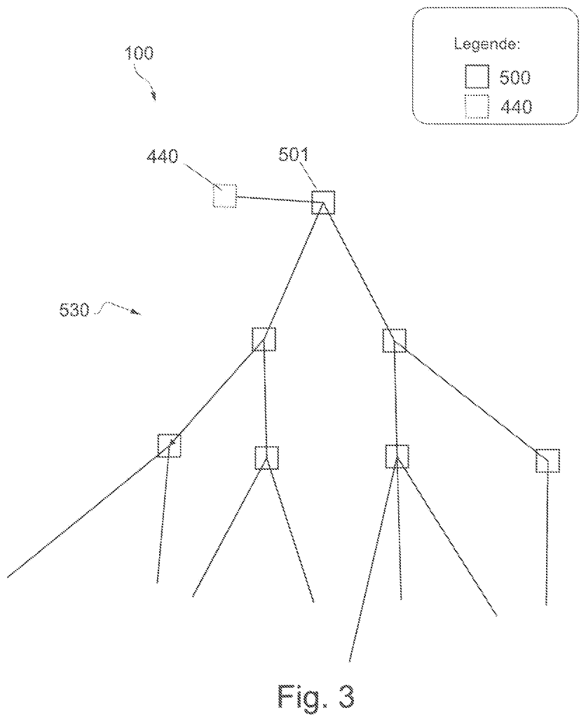

FIG. 3 depicts an example of a device with a tree structure.

Here, FIG. 3 shows a detailed view of a device 100, which is also arranged in an optical waveguide. Corresponding to the illustration in FIG. 2, a plurality of replication regions 500 is shown schematically, a first replication region 501 being optically coupled to an input coupling element 440. Further elements of the plurality of replication region have a tree structure 530.

The replication regions 500 may here be configured both for the transfer of light in the optical waveguide as well as for coupling of light out of the optical waveguide in order to generate an illumination distribution. The degrees of freedom of the illumination distribution that can be generated by a device 100 are further increased by the tree structure.

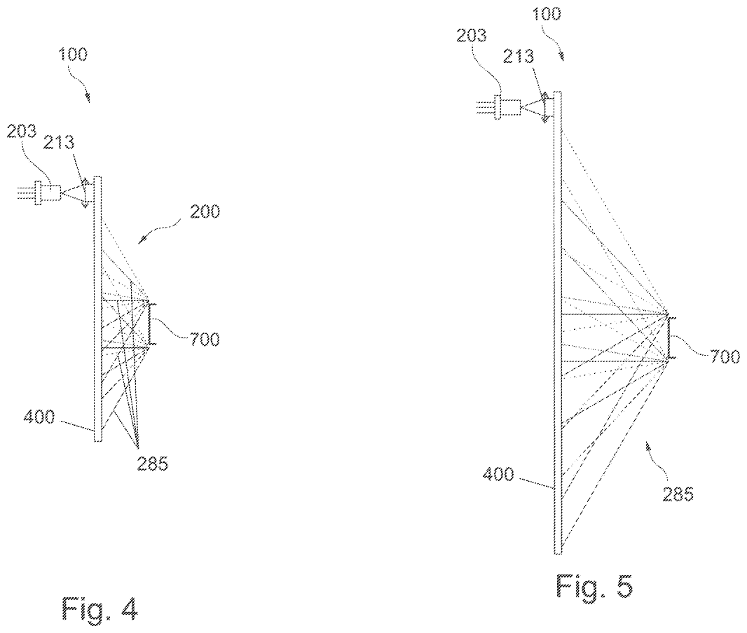

FIG. 4 depicts another exemplary embodiment of FIG. 1A and FIG. 1B.

FIG. 4 shows an exemplary embodiment of a device 100, wherein the reference numerals agree with the devices of FIG. 1A and FIG. 1B. The device 100 of FIG. 4 is configured to provide an illumination distribution 200 to illuminate an object 700. In the exemplary embodiment shown in FIG. 4, the illumination distribution 200 is configured such that a plurality of rays 285 are emitted from different regions of the optical waveguide 400.

In other words, the illumination distribution comprises different light beams, for example the rays 285 which overlap at the waveguide 400. A portion of the waveguide 400 may also be the origin of light beams having different directions. For example, this may be achieved by means of multiply exposed volume holograms that are used as replication regions and/or output coupling elements.

As a result, the received light is effectively focused onto the object 700 by the device 100. In the exemplary embodiment shown, the rays 285 are collimated and are emitted from the optical guide 400 with discrete angular regions.

The optical waveguide 400 may here comprise at least one output coupling element. For example, for each of the plurality of rays 285 a respective output coupling element may be provided. In this case, each of the respective output coupling elements may receive light from several replication regions.

FIG. 5 illustrates another device 100. This device corresponds essentially to the device of FIG. 4. The device of FIG. 5 is also configured to illuminate the object 700 at the same angles as the device 100 in FIG. 4, but at a greater distance between the object 700 and the optical waveguide 400. Accordingly, the device 100 of FIG. 5 is realized larger than the device 100 of FIG. 4.

By comparing FIG. 4 and FIG. 5, it can be seen that it may be necessary to increase the diameter of the waveguide when an object with a given diameter is to be illuminated at a greater distance with the same or a similar illumination distribution.

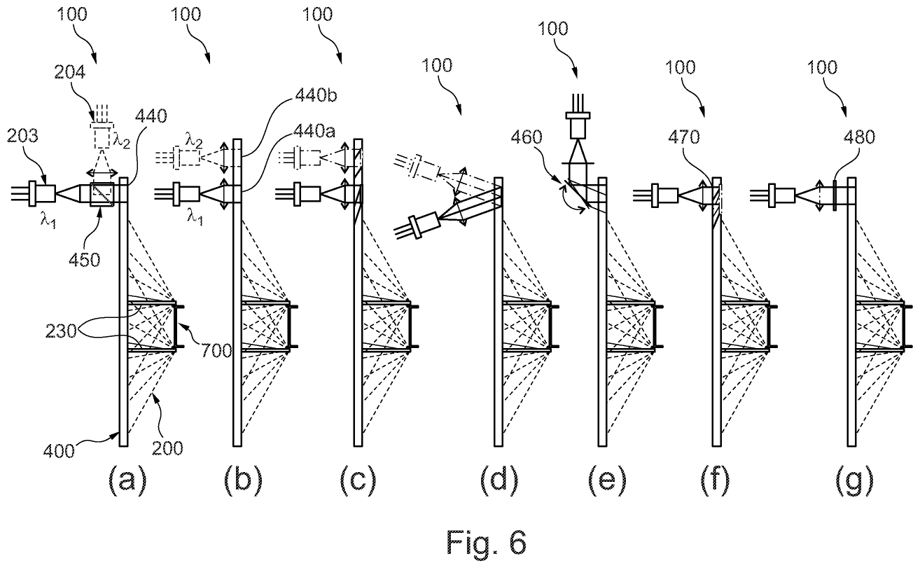

FIG. 6 depicts devices in accordance with various exemplary embodiments, which are multi-channeled and/or switchable.

Here, subfigures FIGS. 6(a) to 6(g) depict different examples of multi-channeled or switchable devices which are configured to provide different illumination distributions.

Various concepts of multi-channel waveguide systems are described below with reference to the devices 100 at (a) to (g). The concepts may utilize high spectral and/or angular selectivity of diffractive elements, for example of volume holograms or other microstructured optical elements in order to be able to transmit several beams of rays independently of one another within the same volume of the optical guide 400. High spectral selectivity refers here to a decrease in the efficiency of the element, for example, by 50% half width, sometimes also known as full width at half maximum (FWHM), with wavelength deviations from the design wavelength, for example <40 nm, for example <10 nm.

High angular selectivity refers to a decrease in the efficiency of the element by 50% FWHM with a deviation of the beam incidence angle from a design angle for which the respective optical element is designed, for example to receive an associated input light beam from this angle, for example <10°, for example <2°. In these cases, but not limited thereto, several beams of rays with different directions and/or wavelengths may propagate within the same volume of the optical waveguide 400 and may be selectively coupled and transferred by associated optical elements, sometimes also described as “matching” optical elements. In other words, selectively acting replication regions may be provided within an identical volume of the optical guide 400. These may function in superposition and convert the light into different illumination distributions for different characteristics, for example angles of incidence. This is sometimes also described as multiplexing, for example as spectral multiplexing, if the optical elements, for example volume holograms, are configured in such a way that they have different coupling behaviors for different spectral properties of the light. Other types of multiplexing are also possible, for example angle-dependent or polarization-dependent multiplexing, as well as combinations thereof.

This basic idea will be briefly explained below using the example of side views of device 100 in FIG. 6. Only a maximum of two light sources are shown here by way of example; this is of course not to be interpreted as restrictive; more complex systems, for example with more than two light sources, are also possible.

The device at (a) depicts a device 100 which is configured to receive light from a first light source 203 with a first wavelength λ1 and light of a second wavelength λ2 from a second light source 204, and to generate a illumination distribution 200 for each received wavelength. In the example shown, the illumination distribution 200 comprises a illumination distribution, which is composed of the illumination distribution 200 of FIG. 4 and a illumination distribution with fixation marks 230. Such a structure may have the advantage that it is possible to use the same optical waveguide 400 in different wavelength ranges to provide different illumination distributions for different purposes, in the example shown, for example, the fixation marks 230 at a wavelength λ2 of the second light source 204 in the visible range and infrared light at a wavelength λ1 of the first light source 203 in the infrared range.

FIG. 6(b) shows an alternative implementation of the device of FIG. 6(a) with a differently configured input coupling element 440. In this embodiment, the input coupling element 440 comprises two different regions, with a first coupling region 440A configured to couple the light from the first light source 203 and a second coupling region 440B configured to couple the light from the second light source 203 into the optical waveguide 400.

FIGS. 6(c) to 6(g) show various possibilities for realizing switchable systems and/or systems that allow to overlay, sometimes also referred as superpose, several illumination distributions.

In the example of FIG. 6(c), the light sources 203, 204 are arranged laterally offset and are coupled into the optical waveguide 400 at different positions by input coupling elements 440A, 440B.

The respective associated input coupling elements 440A, 440B may be designed in such a way that even with light sources 203, 204 of the same type, different couplings into the optical waveguide 400 are achieved, for example different coupling angles. The device 100 can thus be configured to provide two illumination distributions, in the example shown one illumination distribution for each respective light source. In some examples, these illumination distributions may be selected independently of one another, for example on the basis of the previously described angular selectivity and/or wavelength selectivity of the optical elements used.

FIG. 6(d) depicts a variation of FIG. 6(c), the two light sources 204, 203 impinging onto an input coupling element 440 at different angles. This input coupling element is configured to couple the two light sources into the optical waveguide 400 independently of one another. In the example of FIG. 6(e), there is only one light source 203. Here, the angle of incidence of the light from the light source 203 is varied by a scanning mirror 460, which results in a switchable illumination distribution. In the example of FIG. 6(f), a switchable optical element 470, for example a switchable hologram, is present within the optical waveguide 400. This also enables to achieve a superposition of different illumination distributions. In the example of FIG. 6(g), a polarization changing element 480 changes the polarization properties of the light from the light source 203. The optical elements of the device 100 may have polarization-dependent properties, so that different illumination distributions may also be effected by varying the polarization of the light incident onto the device 100.

The examples shown in FIGS. 6(a) to 6(g) may also be combined with one another. For example, different light sources with different polarization directions corresponding to the example in FIG. 6(g) may be combined with a scanning mirror as shown in FIG. 6(e). However, any other combinations of the elements and procedures shown are also possible.

In connection with the following Figures, various possible applications of the devices shown thus far will be illustrated further.

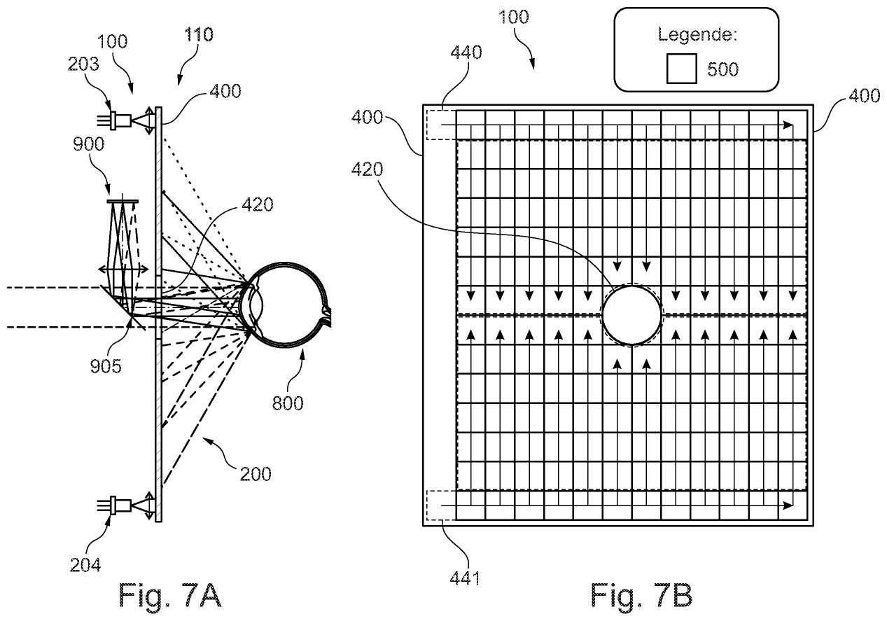

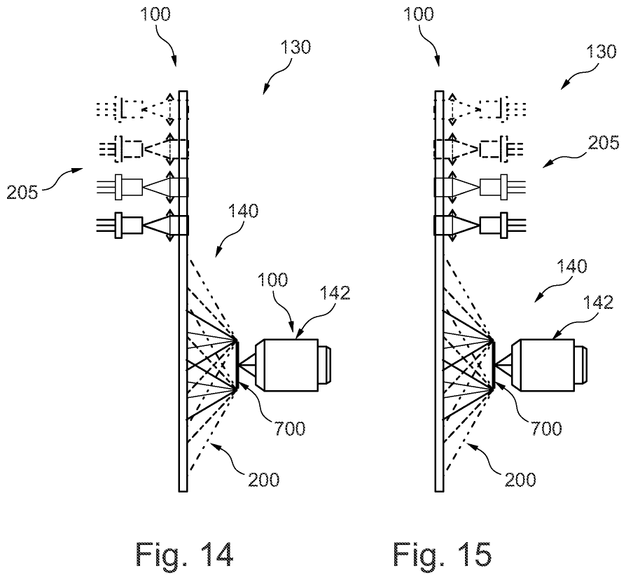

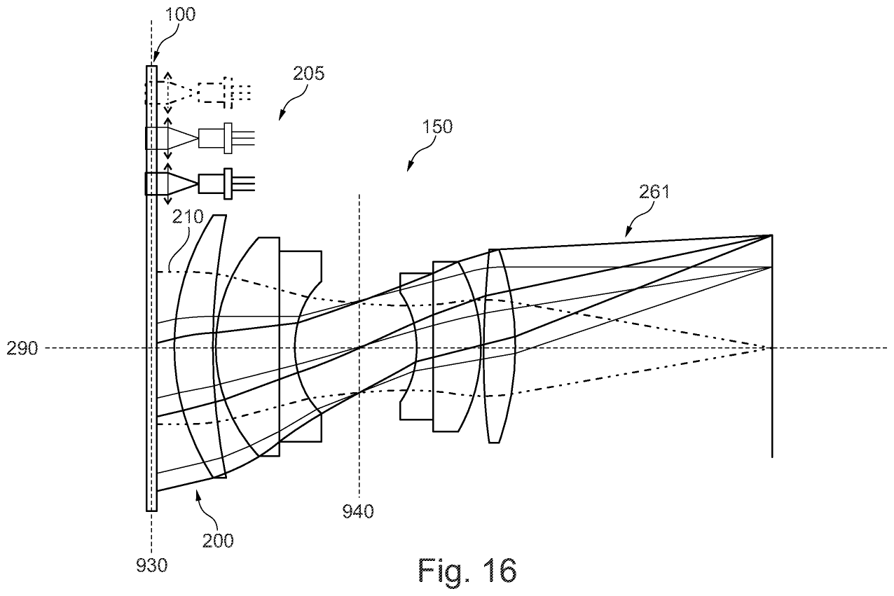

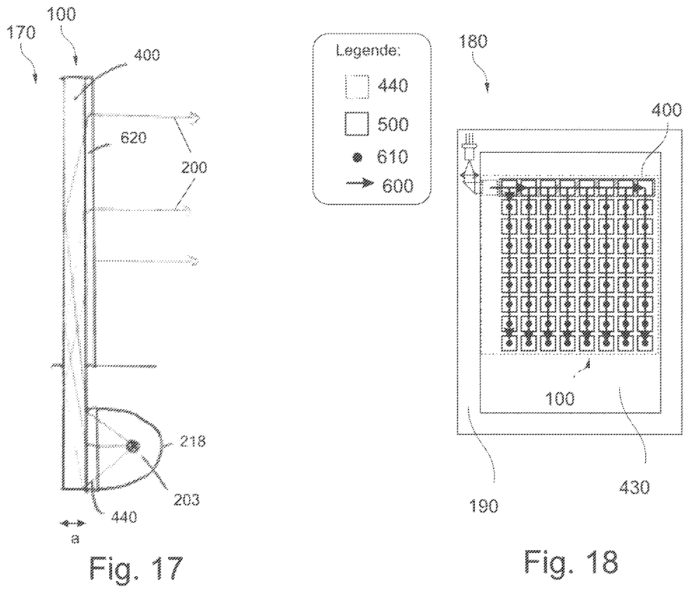

FIG. 7A to FIG. 13B show devices according to the invention which are used to provide a keratometer. FIG. 14 and FIG. 15 depict various structures for a microscope in accordance with various exemplary embodiments. FIG. 16 shows a calibration device for an optical apparatus, FIGS. 17 and 18 show devices for area illumination and a window for a building.

FIG. 7A depicts a side view of a keratometer according to the invention. FIG. 7B depicts a front view.

With a device 100 according to the invention, a illumination distribution 200 for keratometric measurement of the cornea of an eye 800 is provided. The light reflected by the cornea of the eye 800 is detected by a detection device 900 along a detection beam path 905 and may then be analyzed in order to infer the topology of the cornea. The optical waveguide 400 of the device 100 has a cutout 420. In order to achieve an illumination distribution 200 suitable for keratometry, which illuminates the entire eye to be examined as far as possible, despite the cutout 420, the light is provided by two light sources 203, 204 and coupled in by two input coupling elements 440, 441. Based on the respective input coupling elements 440, 441, the light is replicated over a plurality of replication regions and is coupled out in the direction of the eye 800 as an illumination distribution 200.

In the example of the keratometer shown, the surface normal of the optical waveguide is arranged parallel to a main visual axis of the eye 800. In other exemplary embodiments, however, the normal of the optical waveguide may also be arranged barely not parallel to the main visual axis of the eye 800. In this way, for example, reflections can be reduced or avoided.

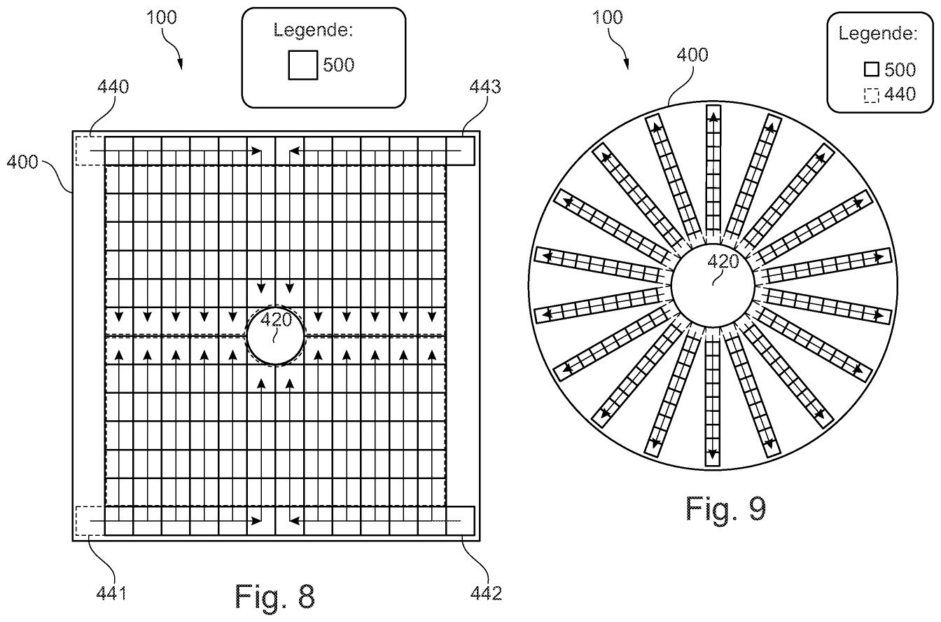

FIG. 8 depicts another exemplary embodiment of a keratometer.

In the device 100 of FIG. 8 there are four input coupling elements 440 to 443. The plurality of replication regions 500 are coupled to one another in such a way that an illumination distribution like the illumination distribution 200 of FIG. 7(a) may be provided by the plurality of replication regions 500.

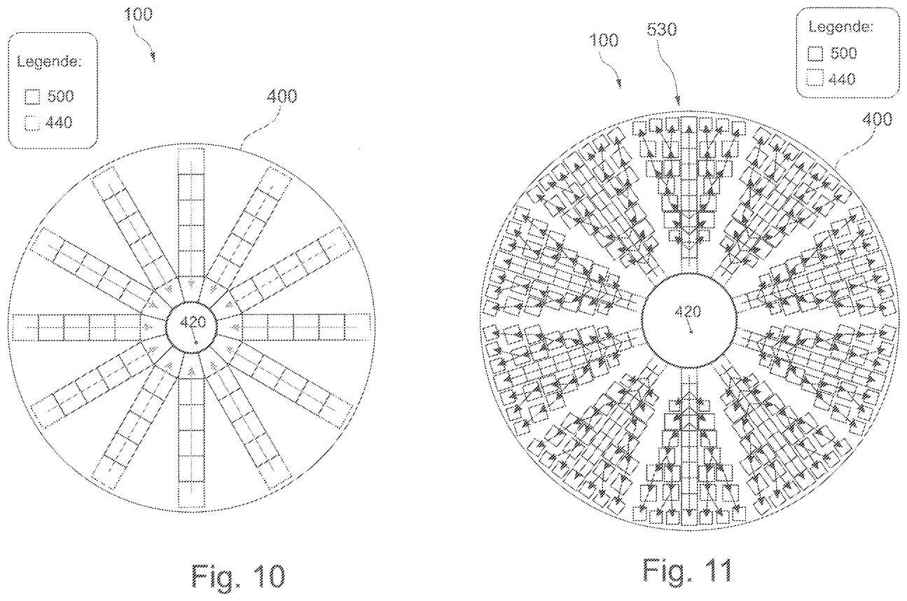

FIG. 9 to FIG. 12 depict various implementations of keratometry devices. The devices of FIGS. 9 to 11 each have a cutout 420 in the center of a round optical waveguide 400. In the example of FIG. 9, the light near the cutout 420 is received by a plurality of input coupling elements 440 and transferred in series to a plurality of replication regions, which likewise serve to couple the light out into the direction of the eye 800. FIG. 10 depicts a similar arrangement, whereby the plurality of input coupling elements 440 is not arranged in spatial proximity to the cutout 420, but in the vicinity of the edge of the optical waveguide 400. The light received by the plurality of input coupling elements 440 is transferred in series to the plurality of replication regions. Here, the replication regions may be shaped arbitrarily. In the example shown in FIG. 10, these regions are rectangular in shape, but in the vicinity of the cutout 420 they have a more complex shape, wherein other shapes are also possible and the shapes shown are only exemplary.

In the exemplary embodiment of FIG. 11, the input coupling elements 440 are again arranged in the vicinity of the cutout 420 in accordance with FIG. 9. The optical coupling of the plurality of replication regions now has a tree structure 530. In this way, homogeneous illumination of the eye can be achieved.

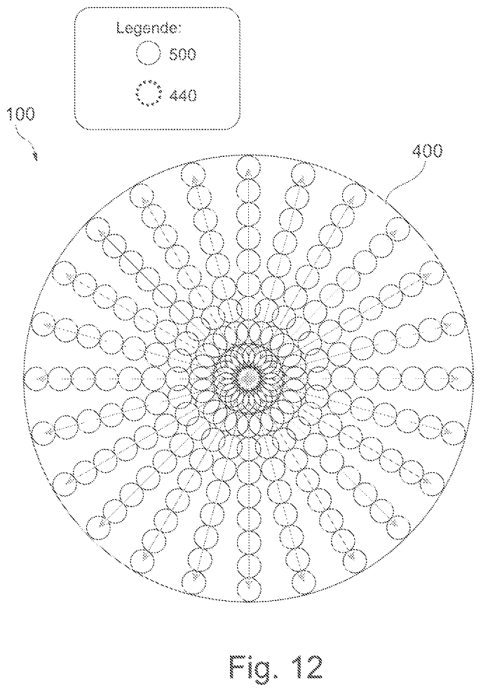

In the exemplary embodiment of FIG. 12, the light is coupled in via a single input coupling element 440 in the center of the optical waveguide 400. The plurality of replication regions 500 are circularly shaped in the exemplary embodiment in FIG. 12 and overlap one another in the front view. This can be achieved by an offset within the optical waveguide or by a volumetric overlap, for example in the case of volume holograms, as already described above. Due to the angular selectivity of some diffractive elements, it may be possible to provide a well-defined light distribution 200 despite the overlap of the plurality of replication regions 500.

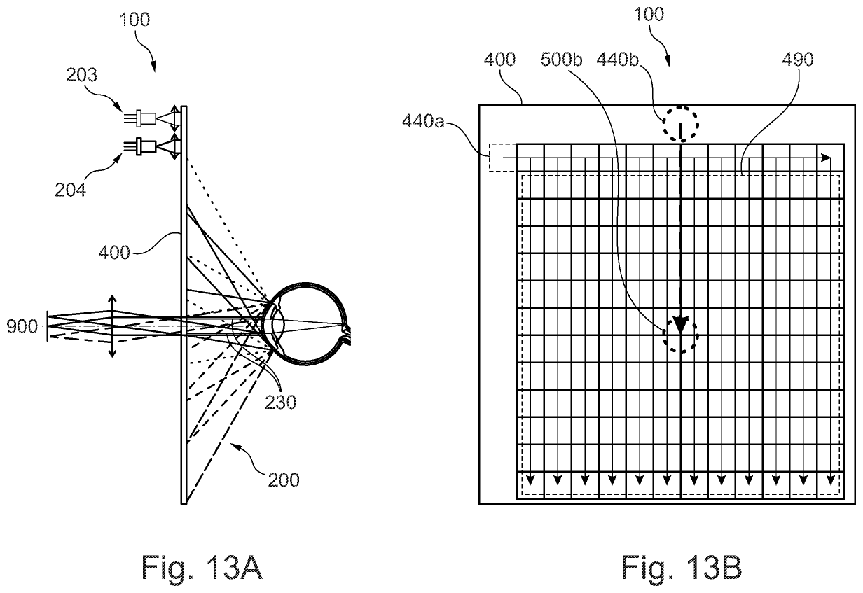

FIG. 13A and FIG. 13B depict an alternative implementation of a keratometer with fixation marks.

In the exemplary embodiment of FIG. 13A and FIG. 13B, the optical waveguide 400 has no cutout. The observation by the detection device 900 takes place through the optical waveguide 400. The keratometer 120 may comprise a device 100 according to an exemplary embodiment of FIG. 6. An exemplary embodiment according to FIG. 6(b) is shown here. As described in connection with FIG. 6(b), the two light sources 203, 204 generate two illumination distributions 200, the first light source 203 providing the infrared illumination distribution 200 required for keratometry, and a light source 204 emitting in the visible range providing fixation marks 230. In the side view of FIG. 13B it can be seen that the device 100 has an input coupling element 440a for the infrared light for this purpose, which acts in accordance with the exemplary embodiment of FIG. 2. For the fixation marks, the light from the light source 204 is coupled in by an input coupling element 440b and is coupled out by a replication region 500b to provide the fixation marks 230 as an illumination distribution.

Another application example from the field of microscopy will be explained below.

FIG. 14 and FIG. 15 depict a microscope in accordance with various exemplary embodiments.

The microscope 130 has a sample illumination device 140 and an eyepiece 142. This illumination device comprises a device 100 according to the previous exemplary embodiments and is configured to generate an illumination distribution on a sample 700. In particular, the illumination distribution may be a pattern on the sample 700. For this purpose, the device 100 may be configured to receive light from a light source 205 which can be modulated in multiple ways. The received light can then be converted into a illumination distribution 200. Here, the light source, which can be modulated in multiple ways, may be arranged on both the side facing away from the microscope, as shown in FIG. 14, but may also be arranged on the side facing towards the microscope, as shown in FIG. 15. In particular, because of the freedom of design of the device 100 an angle-variable illumination of the microscopic sample 700 may be provided. This allows to fulfill illumination requirements in microscopy, such as those occurring in Fourier ptychography, with reduced effort and/or increased quality. The light sources that can be modulated may be switched on a time-selective basis.

Frequently, beams of rays do not have to be switched individually, but beam groups can be switched on and off to accelerate the image acquisition. Each of these groups of jointly switched beams of rays can also be regarded as one illumination distribution. Here, a illumination distribution may be provided from a light source assembly as described above and below. Some image optimization methods may, for example, already be realized with light from 4 separately switchable illumination distributions. For this, however, it is necessary that each of the four switchable illumination distributions sends light onto the sample from several discrete directions. Such switchable illumination distributions, also for fewer or more than 4 switching states, can be provided according to the invention.

Another application example will be described below.

FIG. 16 depicts a calibration device 150 for an optical apparatus 910 in accordance with various exemplary embodiments.

The device according to various exemplary embodiments may advantageously be employed for calibrating and adjusting optical imaging systems, for example lenses. This may be particularly advantageous in connection with optical apparatus that are difficult to access, for example lenses or other imaging systems, which are located inside machines or which are used in difficult environmental conditions, for example under water or in space.

In the embodiment of FIG. 16, a planar optical waveguide system 400 is arranged directly in the beam path 290 of an optical apparatus 910. The device 100 is configured to receive light from a light source 205 that can be modulated in multiple ways, and to provide an illumination distribution. However, the light source 205 that can be modulated in multiple ways may also be a light source that cannot be modulated, for example if only a single illumination distribution is needed, for example a single test image.

This illumination distribution 200 may now be used to carry out the calibration of the optical device 910. For this purpose, in particular, different wavelengths of light from the light source 205 that can be modulated in multiple ways may be provided simultaneously or sequentially in time. Additionally or alternatively, the illumination distribution 200 may be provided in such a way that the light 210 leaves the optical waveguide such that it is incident under well-defined incident light angles into the optical apparatus 910. In this way, the optical apparatus 910 may be calibrated advantageously.

At the same time, due to the high angular selectivity of the optical elements in the optical waveguide 400, the normal operation of the optical apparatus 910 is not or only negligibly influenced. In the exemplary embodiment shown in FIG. 16 the calibration device 150 is located in a first installation position 930. In particular, such an installation position outside of the optical elements of the optical apparatus 910 may enable a simple exchangeable installation, for example as a filter element.

Alternatively or additionally, the calibration device 150 may be installed at a position within optical elements of the optical apparatus 910. This may allow an efficient partial calibration of individual optical elements.

By providing the illumination distribution not in front of, but e.g. between assemblies of the lens, new concepts for testing and adjusting optical apparatus can be implemented. In such cases, the illumination distribution may, for example, represent the nominal wavefront of the optical apparatus that would arise through the upstream subgroups of lenses and a standard test object.

The installation of these calibration devices such as the calibration device 150 shown may be permanent or temporary. For example, the calibration device may be moved into the beam path for calibration. In other embodiments, it may also remain permanently in the beam path. Here it may be advantageous that, due to the strong wavelength and/or angular selectivity of the devices used, the influence of the calibration device on the beam path of the optical apparatus may be small. In those cases where the device is permanently installed in the beam path, the device can be taken into account in the optical design of the optical apparatus. Due to the high angular and spectral selectivity of the device in the optical waveguide, only narrow spectral subbands may be filtered out by an optical waveguide for a selected field point of the optical apparatus, such that the functionality of the optical apparatus is not or only minimally influenced. At these wavelength bands, the adjustment marks and test patterns may be fed in by reflection with high efficiency.

The test patterns offered by the device may be displayed in different distances, wavelengths, positions, and shapes. Thereby it is possible to generate several test patterns at the same time with one radiation source. However, several light source assemblies may also be used and/or others of the described procedures may be applied to generate switchable patterns additionally or alternatively.

FIG. 17 depicts an area lamp 170 comprising a device 100 in accordance with various exemplary embodiments. In the exemplary embodiment of FIG. 17, light is provided from a first light source 203 from a parabolic reflector 218 to an input coupling element 450 of the device 100. As described above, the coupled-in light is transferred by means of a plurality of replication regions within the optical waveguide 400. In the exemplary embodiment in FIG. 17, the illumination distribution 200 is provided by an output coupling element 620. However, other possibilities for providing the light distribution 200, as described above, may also be used. In particular, very small dimensions A of the optical waveguide 400 may be realized here, with a high degree of freedom from the illumination distribution 200 being available at the same time. In the exemplary embodiment depicted in FIG. 17, collimated light is provided as a illumination distribution 200, but other, more complex illumination distributions may also be generated.

FIG. 18 depicts a window 180 in accordance with various exemplary embodiments. The window 180 comprises window glass 430, with an optical waveguide 400 according to various exemplary embodiments of the device described above. The window 180 further comprises a window frame 190. A light source 203 is arranged within the window frame 190 and not visible in the exemplary embodiment in FIG. 18. The light source 203 is configured to provide infrared light and to provide this to the at least one input coupling element 450 of the device 100. The device 100 in the optical waveguide generates an illumination distribution 200. In this case, the illumination distribution 200 may be provided as a heat source, for example as a heater for a room in a house, on at least one side of the window. In particular, the infrared light provided by the light source 203 may have a maximum intensity in a spectral range from 1 to 10 μm.

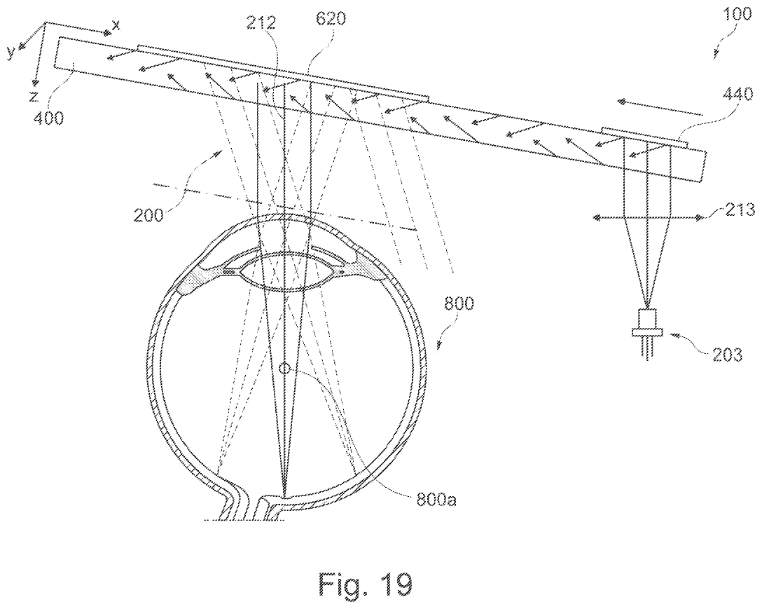

FIG. 19 depicts an illumination device for an active eye implant in accordance with an exemplary embodiment.

The device 100 is configured to provide light from a light source 203 to an active eye implant in the eye 800 of a user. In this application example, the light is coupled into the optical waveguide 400 by an input coupling element 440. The optical waveguide 400 is arranged diagonally opposite the eye. This may offer aesthetic advantages if the optical waveguide is arranged in a pair of glasses.

The light propagates within the optical waveguide 440 in total reflection and is coupled put by an output coupling element 620 and provides the illumination distribution 200 to the eye and thus to the eye implant. In the example shown, the light is provided as a plurality of collimated rays, for example as a collimated beam of rays 212. Here, the plurality of collimated rays are effectively focused, since they pass, originating from the optical waveguide 400, through a larger exit area at the optical waveguide than in an imaginary focusing plane (indicated as a dash-dotted line in front of the eye 800).

Due to the plurality of coupled out rays, it can also be ensured, when the eye is rotated about the eye pivot point 800a, that the eye implant is supplied with light regardless of the viewing direction.

As already mentioned, the above exemplary embodiments are merely for illustrative purposes and are not to be construed as limiting. In particular, exemplary embodiments may also be combined with one another, partially as well. For example, teachings described as exemplary embodiments in connection with the microscopy application may also be used in connection with general illumination devices, but also in other exemplary embodiments, for example in connection with the exemplary embodiment of the window, when the window is to heat a specific object. As another example, the fixation marks described in connection with the keratometer may also be used to provide fixation marks in a calibration device or a microscope.

1. A device for generating a luminous distribution for illuminating an object, comprising:
an optical waveguide comprising the following optical elements:
at least one input coupling element configured to couple light into the optical waveguide as a light beam having an associated beam profile,
a plurality of replication regions for replication of the light beam, each configured to receive at least one associated input light beam having an input beam profile and to provide a plurality of associated output light beams having respective output beam profiles,
wherein at least one first replication region of the plurality of replication regions is optically coupled with a second replication region of the plurality of replication regions, such that the second replication region is configured to receive at least one of the plurality of associated output light beams of the first replication region as the associated input light beam of the second replication region, and
wherein the first replication region is optically coupled with the at least one input coupling element for receiving the light beam as the associated input light beam of the first replication region,
the device being configured to couple emitted light from a number of the plurality of replication regions out of the optical waveguide to provide the luminous distribution.

2. The device according to claim 1, wherein the optical waveguide is configured to receive the light having a first modulation, the device being configured such that the luminous distribution has a second modulation, the second modulation having a greater number of extrema than the first modulation.

3. The device according to claim 1, wherein the device does not comprise a spatial light modulator configured to modulate, on the basis of data, light to be coupled into the optical waveguide.

4. The device according to claim 1, wherein at least a subset of the plurality of replication regions provides a partial luminous distribution of the luminous distribution, said partial luminous distribution having effective focusing.

5. The device according to claim 1, wherein the luminous distribution comprises different light beams overlapping at the optical waveguide.

6. The device according to claim 1, wherein at least one of the optical elements is a volume hologram, said volume hologram being positioned straight or at an angle within the optical waveguide and/or multiply exposed.

7. The device according to claim 1, wherein the device comprises a light source assembly, the light source assembly being configured to provide the light and comprising at least one of the following elements:
two light sources configured to provide light in different directions and/or in different wavelength ranges and/or to different illumination positions of the at least one input coupling element,
a beam splitter,
a scanning mirror,
a switchable element.

8. The device according to claim 1, wherein the plurality of replication regions comprises a first set of replication regions, which are each optically coupled to one another, and the replication regions of the first set of replication regions being each configured to:
provide at least one first associated output beam of the plurality of output light beams to another replication region of the first set of replication regions, and
not provide at least one second associated output beam of the plurality of output light beams to another replication region of the first set of replication regions, to obtain a number of emitted beams of the first set of replication regions.

9. The device according to claim 6, wherein the optical coupling has a serial structure.

10. The device according to claim 8, wherein the optical coupling has a tree structure.

11. The device according to claim 8, wherein the plurality of replication regions comprises a second set of replication regions, which are each optically coupled to each other and a subset of which is configured to receive the number of emitted beams of the first set of replication regions as respective input light beams.

12. The device according to claim 11, wherein the optical coupling of the first set of replication regions and/or the second set of replication regions comprises an optical coupling in series and/or the optical coupling comprises a tree structure.

13. The device of claim 1 wherein the optical elements further comprise:
at least one output coupling element configured to couple light out of the optical waveguide.

14. The device according to claim 13, wherein the at least one output coupling element and/or the at least one input coupling element comprise one or more other optical elements selected from a group comprising: a lens, a prism, a surface grating, a polarization filter.

15. The device according to claim 4, wherein the luminous distribution is configured such that a plurality of rays from different regions of the optical waveguide are emitted such that the emitted light is effectively focused and/or effectively defocused.

16. The device according to claim 15, wherein the plurality of rays are collimated and/or emitted from the optical guide in discrete angular regions.

17. The device according to claim 13, wherein the at least one output coupling element comprises at least one other optical element configured to generate a pattern of coupled out light.

18. The device according to claim 1, wherein at least one of the optical elements and/or the at least one other optical element is selected from: a diffractive element, a switchable diffractive element, a volume hologram.

19. The device according to claim 1, wherein the at least one input coupling element is configured to perform coupling based on a characteristic of the light, and wherein the replication regions are configured to produce at least two different associated luminous distributions for at least two different characteristics of the light.

20. The device according to claim 1, wherein the device is configured to provide a luminous distribution for illuminating, at variable angles, an object remote from the device, the object having a smaller diameter than the optical waveguide.

21. The device according to claim 20, wherein the device is configured to provide the luminous distribution for the object when the object is located at an angle to a surface normal of the optical waveguide.

22. An optical waveguide system having a plurality of optical waveguides, according to claim 1, the plurality of optical waveguides having a common optical waveguide with an output coupling area,
wherein the common optical waveguide has at least one cutout with a cutout area in the output coupling area, and wherein the plurality of devices is arranged such that the luminous distributions of the plurality of devices originate from at least 80% of the output coupling surface without the cutout area.

23. A device according to claim 1, wherein the optical waveguide has first and second sides, and wherein the luminous distribution comprises a first luminous distribution on the first side and a second luminous distribution on the second side of the optical waveguide.

24-41. (canceled)
